(19) United States
(12) Patent Application Publication
(10) Pub. No.: US 2021/0321890 A1
(43) Pub. Date: Oct. 21, 2021

(54) Apparatus and Method for Determining Fetal Movement

(71) Applicant: Owlet Baby Care Inc., Lehi, UT (US)

(72) Inventors: Ajay Iyer, Peabody, MA (US); Vikranth Siddenki, San Diego, CA (US); Steven Reeves, American Fork, UT (US)

(21) Appl. No.: 17/230,375

(22) Filed: Apr. 14, 2021

Related U.S. Application Data
(60) Provisional application No. 63/010,258, filed on Apr. 15, 2020.

(51) Int. Cl. A61B 5/024; A61B 5/344; A61B 5/00; A61B 5/366

(52) U.S. Cl. CPC A61B 5/02411; A61B 5/344; A61B 5/7225; A61B 5/7221; A61B 5/7257; A61B 5/366; A61B 5/7267

(57) ABSTRACT

A technology for detecting fetal movement. In one example an electrocardiogram (ECG) dataset can be obtained where the ECG dataset contains fetal heart rate (FHR) values acquired from a pregnant subject, and the ECG dataset is for a segment of time during which an FHR is monitored using an ECG monitor. An FHR baseline can be calculated for the FHR values in the ECG dataset, and the ECG dataset can be analyzed to identify an accelerated FHR value that exceeds the FHR baseline that is followed in time in the ECG dataset by a decelerated FHR value that is less than or equal to the FHR baseline. Identifying the accelerated FHR value followed in time in the ECG dataset by the decelerated FHR value in the ECG dataset may indicate a fetal movement.

### BACKGROUND

Non-invasive health monitoring devices are increasingly helping people to better monitor their health status both at an activity/fitness level for self-health tracking and at a medical level providing more data to clinicians with a potential for earlier diagnostic and guidance of treatment. Some consumer wearable devices have incorporated pulse-oximetry as a sensor for gathering biometric data. While pulse-oximetry data can provide several important insights into an individual's health, there is a desire for increased monitoring that provides information beyond what a standard pulse-oximeter is able to provide. For example, standard wrist-worn pulse-oximeters are unable to provide health information for a fetus of a pregnant woman. One potential means for providing fetal health information is through the use of abdominal electrodes for fetal monitoring. Abdominal electrodes for fetal monitoring are non-invasive and can potentially allow for in-home and ambulatory use.

### SUMMARY

An apparatus/system and method is described for determining fetal movement. The apparatus/system may include at least one processor, a memory device including instructions that, when executed by the at least one processor, cause the system to obtain an electrocardiogram (ECG) dataset containing fetal heart rate (FHR) values acquired from a pregnant subject, wherein the ECG dataset is for a segment of time during which an FHR is monitored using an ECG monitor. The instructions that, when executed by the at least one processor, may cause the system to calculate an FHR baseline for using the FHR values in the ECG dataset. The instructions that, when executed by the at least one processor, may cause the system to analyze the ECG dataset to identify an accelerated FHR value that exceeds the FHR baseline that is followed by and a decelerated FHR value that is less than or equal to the FHR baseline, wherein the accelerated FHR value occurs before the decelerated FHR value. The instructions that, when executed by the at least one processor, may cause the system to determine that the accelerated FHR value followed in time by the decelerated FHR value in the ECG dataset indicates a fetal movement.

A method is described for determining fetal movement. The method may include obtaining electrocardiogram (ECG) data from a pregnant subject, wherein the ECG data contains maternal and fetal heart rate information. The method may include inputting the ECG data to an artificial neural network model trained to predict which heart rate heart rate values in the ECG data are fetal heart rate (FHR) values, wherein the artificial neural network model includes a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to FHR predictions. The method may include generating an ECG dataset of FHR values from the FHR predictions output by the artificial neural network model. The method may include calculating an FHR baseline using the FHR values in the ECG dataset. The method may include analyzing the ECG dataset to identify an indication of fetal movement defined by an accelerated FHR value which is followed in time by a decelerated FHR value in the ECG dataset, wherein the accelerated FHR value exceeds the FHR baseline, and the decelerated FHR value is less than or equal to the FHR baseline.

A non-transitory machine-readable storage medium including instructions embodied thereon is provided. The instructions, when executed by at least one processor may obtain electrocardiogram (ECG) data from a pregnant subject, wherein the ECG data contains maternal and fetal heart rate information. The instructions, when executed by at least one processor may input the ECG data to an artificial neural network model trained to predict which heart rate heart rate values in the ECG data are fetal heart rate (FHR) values, wherein the artificial neural network model includes a first series of convolutional layers to separate a fetal ECG signal from a maternal ECG signal, a fast Fourier transform (FFT) layer to convert the fetal ECG signal to an ECG frequency representation, and a dense layer to decode the ECG frequency representation to a FHR prediction. The instructions, when executed by at least one processor may generate an ECG dataset of FHR values from the FHR predictions output by the artificial neural network model. The instructions, when executed by at least one processor may calculate an FHR baseline using the FHR values in the ECG dataset. The instructions, when executed by at least one processor may analyze the ECG dataset to identify an indication of fetal movement defined by an accelerated FHR value which is followed in time by a decelerated FHR value in the ECG dataset, wherein the accelerated FHR value exceeds the FHR baseline, and the decelerated FHR value is less than or equal to the FHR baseline

### BRIEF DESCRIPTION OF THE DRAWINGS

FIG. 1 is a block diagram illustrating an example of a processing system used to generate a fetal movement probability.

FIG. 2 is a flow diagram that illustrates an example method for detecting fetal movement via analysis of a fetal heart rate to identify a fetal heart rate acceleration and corresponding deceleration indicating a fetal movement.

FIG. 3 a block diagram illustrating an example artificial neural network model configured to extract a fetal heart rate from an ECG signal.

FIG. 4 is a flow diagram that illustrates an example method for preprocessing ECG data.

FIG. 5 is a block diagram illustrating an example processing system which can incorporate prior FHR predictions to determine a fetal heart rate probability.

FIG. 6 is a block diagram illustrating an example network architecture for an artificial neural network model which incorporates prior FHR information to generate a fetal heart rate probability.

FIG. 7 is a block diagram that illustrates an example artificial neural network model configured to generate peak probabilities that correspond to individual heart rate peaks which can be decoded to produce a final fetal heart rate prediction.

FIG. 8 is a block diagram illustrating an example artificial neural network model trained using prior FHR predictions.

FIG. 9 is a flow diagram that illustrates an example method for obtaining a fetal heart rate from an ECG signal.

FIG. 10 is block diagram illustrating an example of a computing device that may be used to execute a method for generating a fetal movement probability.

### DETAILED DESCRIPTION

Technologies are described for detecting fetal movement by analyzing a fetal heart rate to identify a fetal heart rate acceleration and corresponding deceleration that is indicative of a fetal movement. A fetal heart rate can be extracted from an electrocardiogram (ECG) signal acquired from an array of electrodes (or other data collecting device) in contact with a maternal abdomen, and the fetal heart rate can be analyzed to identify an accelerated fetal heart rate which is followed by a decelerated fetal heart rate. The accelerated fetal heart rate and the subsequent decelerated fetal heart rate may indicate a movement by a fetus. Fetal movement refers to motion of a fetus caused by the fetus' own muscle activity. Fetal locomotor activity may begin during the late embryological stage and continue throughout fetal development.

In one example, determining a fetal movement can include obtaining an ECG dataset containing fetal heart rate (FHR) values from a pregnant subject (i.e., a person who is carrying a developing fetus within the person's body). The ECG dataset can be for a segment of time (e.g., 5, 8, 10 minutes) during which a FHR is monitored using an ECG monitor. In one example, the ECG dataset can be generated using an artificial neural network model that extracts fetal heart rate values from an ECG signal and outputs an ECG dataset of FHR values. The FHR values in the ECG dataset can be evaluated to determine whether a sufficient portion of the FHR values meet a quality threshold (e.g., at least 30%, 40%, 45%, 50%, 60%, etc. of FHR values in the ECG dataset are quality FHR values) that allows a FHR baseline (e.g., average or nominal FHR) to be calculated.

In the case that the quality of the FHR values identified in the ECG dataset meets the quality threshold, a FHR baseline can be calculated using the FHR values in the ECG dataset. In some examples, a baseline offset can be added to the FHR baseline. Thereafter, the ECG dataset can be analyzed to identify an accelerated FHR value that exceeds the FHR baseline. If the accelerated FHR value is identified, the position of the accelerated FHR value in the ECG dataset can be marked as a possible accelerated FHR. The ECG dataset can then be analyzed to determine whether a decelerated FHR value that is less than or equal to the FHR baseline follows the possible accelerated FHR in the ECG dataset. For example, an ECG monitor may output a FHR rate every one second, and the FHR values in the ECG dataset may represent one second of FHR data. Accordingly, the ECG dataset may be a time series dataset having a series of FHR values indexed in time order (i.e., a sequence of discrete-time FHR data).

In the event that a decelerated FHR value follows in time the possible accelerated FHR, the possible accelerated FHR in the ECG dataset can be flagged as an identified accelerated FHR. In one example, as part of determining whether a possible accelerated FHR can be flagged as an identified accelerated FHR, the quality of FHR values located between the possible accelerated FHR and the decelerated FHR value in the ECG dataset can be evaluated, and if a sufficient portion of the FHR values (e.g., more than 50%) have a quality that meets a predetermined quality threshold, then the possible accelerated FHR can be flagged as an identified accelerated FHR. Also, in one example, as part of determining whether a possible accelerated FHR can be flagged as an identified accelerated FHR, a determination may be made whether a duration of time between the possible accelerated FHR and the decelerated FHR value in the ECG dataset is within a time bound defined as a fetal movement. If the time between the possible accelerated FHR and the decelerated FHR value is within the time bound, then the possible accelerated FHR can be flagged as an identified accelerated FHR. The identified accelerated FHR can then be output as corresponding to a detected fetal movement.

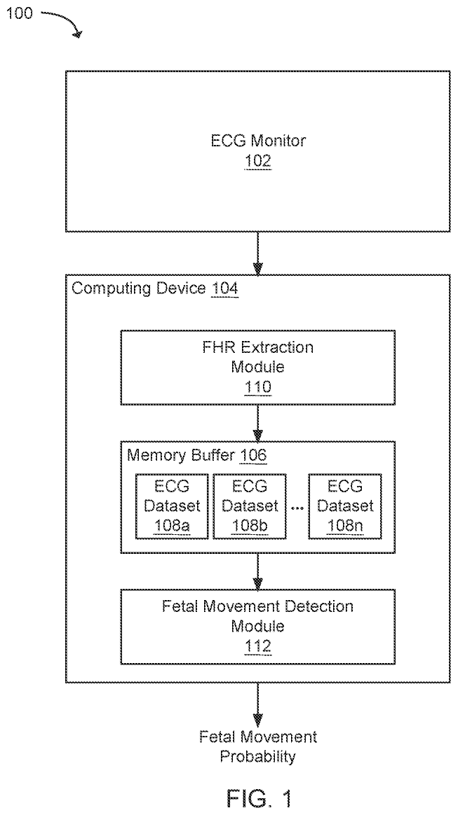

To further describe the present technology, examples are now provided with reference to the figures. FIG. 1 is a block diagram illustrating a high-level example of a processing system 100 used to generate a fetal movement probability. As illustrated, the system 100 can include an ECG monitor 102 that is in communication with a computing device 104 that includes an FHR extraction module 110 and a fetal movement detection module 112. The ECG monitor 102 may be worn by a pregnant subject, and an ECG signal can be obtained from the maternal abdomen of the pregnant subject. The ECG signal can include ECG information for both the subject and the subject's fetus (i.e., ECG data generated by an ECG monitor can include maternal heart rate information and fetal heart rate information). As an illustration, electrodes can be attached to the maternal abdomen of the pregnant subject and the electrical signals of the subject's and the fetus' hearts can be recorded.

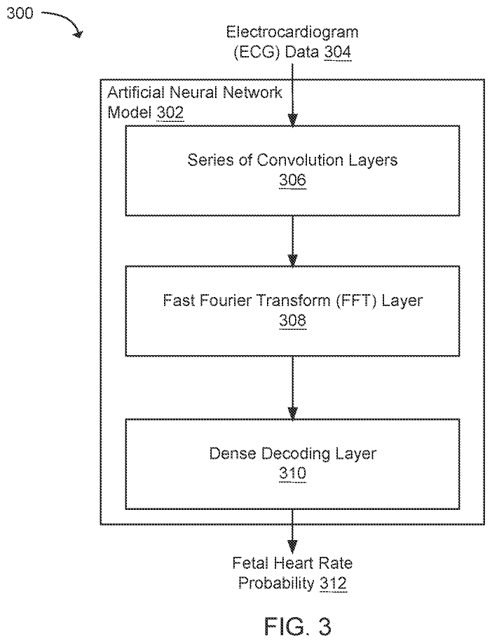

The FHR extraction module 110 may be configured to receive an ECG signal from the ECG monitor 102 and extract a fetal heart rate from the ECG signal. Because the ECG signal obtained from the maternal abdomen of the pregnant subject includes artifacts of a maternal ECG signal, the FHR extraction module 110 can be configured to remove the maternal ECG signal artifacts (as well as other artifacts) from the ECG signal to isolate the fetal ECG signal and determine the fetal heart rate of the fetus. As described in detail later in association with FIG. 3, an artificial neural network model can be trained to predict which values in an ECG signal obtained from a pregnant subject are FHR values. The FHR values extracted from the ECG signal can form one or more ECG datasets 108a-n containing FHR values captured during a segment of time during which a fetal heart rate is monitored using the ECG monitor 102. The ECG datasets 108a-n can be stored in a memory buffer 106 (e.g., a portion of a computer memory reserved for storing ECG data) for a defined amount of time (e.g., 1 minute, 5 minutes, 10 minutes, etc.).

As an example, at specific intervals, the FHR extraction module 110 may extract an FHR value from an ECG signal and store the FHR value in a memory buffer 106 with other FHR values included in an ECG dataset 108a-n. At the end of a defined time segment, the FHR extraction module 110 may close the ECG dataset 108a-n to allow the FHR values in the ECG dataset 108a-n to be processed for the purpose of detecting fetal movement. After closing the ECG dataset 108a-n, the FHR extraction module 110 may create a new ECG dataset 108a-n in a memory buffer 106 and add FHR values extracted from the ECG signal to the new ECG dataset 108a-n. As a non-limiting example, at an interval of every one (1) second, the FHR extraction module 110 may extract an FHR value from an ECG signal (e.g., a 7-channel ECG signal) received from the ECG monitor 102 and add the FHR value to an ECG dataset 108a-n stored in the memory buffer 106. At the end of every ten (10) minute segment, the FHR values in the ECG dataset 108a-n can be provided to a fetal movement detection module 112 to determine whether the FHR values indicate a fetal movement.

In one example, the FHR extraction module 110 may determine a quality of an FHR value extracted from an ECG signal, and FHR extraction module 110 may store the FHR value and the quality of the FHR value (FHR quality) in an ECG dataset 108a-n stored in the memory buffer 106. The quality of an FHR value refers to a confidence in the accuracy of the FHR value to represent a detected fetal heart rate. For example, as describe in more detail later, a machine learning algorithm can be used to analyze an ECG signal and predict which of the heart rate values in the ECG are FHR values. The machine learning algorithm can create a probability distribution of FHR values that indicate a maximum likelihood of a FHR value and provide a quality of a FHR value based on the probability distribution. In one example, the accuracy of the predicted FHR value can be evaluated based on previous fetal heart rate predictions, and a quality of the FHR value prediction can be determined based on a closeness of the FHR value prediction to previous FHR value predictions or surrounding FHR value predictions. For example, a FHR value that is close in value to nearby FHR values may be treated as being more accurate, and a FHR value that is far from the value of nearby FHR values (e.g., an outlier value) may be treated as being less accurate. Accordingly, the FHR extraction module 110 may evaluate an accuracy of a FHR value and assign a quality designation (e.g., a value, weight, symbol, etc.) to the FHR value, and the FHR extraction module 110 may add both the FHR value and the quality designation of the FHR value to an ECG dataset 108a-n.

As mentioned above, at the end of a defined time segment, the FHR extraction module 110 may close an ECG dataset 108a-n, and the ECG dataset 108a-n can be provided to the fetal movement detection module 112. The fetal movement detection module 112 can evaluate the FHR values in the ECG dataset 108a-n to determine whether the ECG dataset 108a-n contains accelerated FHR values that correspond to fetal movements. In one example, the fetal movement detection module 112 may perform preprocessing of an ECG dataset 108a-n to determine whether a quality of the FHR values in the ECG dataset 108a-n meet a quality threshold that allows processing of the ECG dataset 108a-n to detect fetal movement. For example, the fetal movement detection module 112 may evaluate the quality of individual FHR values contained in the ECG dataset 108a-n, and if a sufficient portion (e.g., at least 30%, 50%, 60%, or 70%) of the FHR values in the ECG dataset 108a-n meet the quality threshold, the fetal movement detection module 112 may proceed with processing the ECG dataset 108a-n. The quality of an FHR value can be based on an FHR probability output by the artificial neural network for the FHR value. For example, an output layer of the neural network model may be a softmax layer that has a neuron node for each fetal heart rate value, and the softmax layer may output a heart rate probability map that provides an FHR probability for each heart rate value in an ECG dataset 108a-n. An FHR probability (e.g., a value greater than zero) assigned to an FHR value can indicate or represent the quality or validity of the FHR value (e.g., a probability that a heart rate value in the ECG dataset is a fetal heart rate value). The quality threshold may be a percentage or amount of FHR values in an ECG dataset 108a-n that meet an FHR quality requirement. As a non-limiting example, if at least two (2) minutes, five (5) minutes, or seven (7) minutes of FHR value data in an ECG dataset 108a-n (e.g., ten (10), twelve (12), fifteen (15) minute ECG dataset segment) are determined to be quality FHR values (e.g., FHR values having an FHR probability of at least 50%), then the ECG dataset 108a-n can be processed. The FHR extraction module 110 may consider an FHR value to be valid when a quality of the FHR value is greater than the quality threshold. In the case that a sufficient portion of FHR values in an ECG dataset 108a-n does not meet the quality threshold, the fetal movement detection module 112 may not evaluate the ECG dataset 108a-n to detect fetal movement.

In determining that a sufficient portion of FHR values in an ECG dataset 108a-n meets a quality threshold, the fetal movement detection module 112 may process the FHR values in the ECG dataset 108a-n to estimate an FHR baseline for the FHR values used to determine whether the ECG dataset 108a-n contains FHR accelerations that correlate with fetal movements. For example, the fetal movement detection module 112 can calculate an FHR baseline using the FHR values in the ECG dataset 108a-n. The FHR baseline may be an average the FHR values, which can be calculated by totaling the FHR values and dividing the resulting sum by the number of FHR values included in the ECG dataset 108a-n. The FHR baseline, in one example, may be rounded to the nearest five (5) BPM.

After calculating an FHR baseline, the fetal movement detection module 112 may analyze the ECG dataset 108a-n to identify an accelerated FHR value that exceeds the FHR baseline and is followed in time in the ECG dataset 108a-n by a decelerated FHR value that is less than or equal to the FHR baseline. In one example, a baseline offset (e.g., 2 BPM, 3 BPM, 5 BPM, etc.) can be added to the FHR baseline. The baseline offset can be used to avoid flagging minor fluctuations in an FHR as an accelerated FHR value. Accordingly, an FHR value may need to exceed the baseline offset to be marked as a potential FHR acceleration. As a non-limiting example, the fetal movement detection module 112 may process an ECG dataset 108a-n containing a ten (10) minute segment of six hundred (600) FHR values. The fetal movement detection module 112 may sequentially process the FHR values from the first FHR value in the ECG dataset 108a-n to the six hundredth FHR value in the ECG dataset 108a-n to determine whether an FHR value is greater than the FHR baseline, and a preceding FHR value is less than or equal to the FHR baseline. That is, the fetal movement detection module 112 attempts to identify a position in the ECG dataset 108a-n where an FHR value vector crosses the FHR baseline in an upward direction. In one example, as part of processing the FHR values, the fetal movement detection module 112 may increment a quality counter for every FHR value that meets the quality threshold to allow the quality counter to be used to determine a quality of an accelerated FHR value, as described below.

In the case that the fetal movement detection module 112 identifies an accelerated FHR value that is followed in time in the ECG dataset 108a-n by a decelerated FHR value, the fetal movement detection module 112 may perform one or more validation checks to confirm that the detected accelerated FRH corresponds to a fetal movement.

In one example, the fetal movement detection module 112 may perform a validation check to determine whether a quality (FHR quality) of an accelerated FHR value and the FHR value that immediately precedes the accelerated FHR value in the ECG dataset meet a predetermined quality threshold (e.g., an FHR probability greater than zero). If the quality of both FHR values meet the quality threshold, the fetal movement detection module 112 may mark a position of the accelerated FHR value in the ECG dataset 108a-n as a possible accelerated FHR, and evaluate the quality of intervening FHR values located between the accelerated FHR value and the decelerated FHR value to determine whether a sufficient portion of the intervening FHR values meet the quality threshold. In the case that a sufficient portion of the intervening FHR values meet the quality threshold, the fetal movement detection module 112 may flag the position of the accelerated FHR value in the ECG dataset 108a-n as an identified accelerated FHR and output an indication (e.g., a message to a display) that a fetal movement has been detected.

In another example, the fetal movement detection module 112 may perform a validation check to determine whether an accelerated FHR value is followed in time in the ECG dataset 108a-n by a subsequent accelerated FHR value, indicating that the first detected accelerated FHR value is not an anomaly. For example, after identifying a possible accelerated FHR value in an ECG dataset 108a-n as described above, the fetal movement detection module 112 may continue to process FHR values in the ECG dataset 108a-n to determine whether the ECG dataset 108a-n contains a subsequent accelerated FHR value that is within a time threshold (e.g., 5 seconds, 10 seconds, 20 seconds, 30 seconds, etc.) of the position of the accelerated FHR value marked in the ECG dataset 108a-n. If a subsequent accelerated FHR value is identified as being within the time threshold, the fetal movement detection module 112 may flag the position of the possible accelerated FHR value as an identified accelerated FHR and output an indication that a fetal movement has been detected.

In another example, the fetal movement detection module 112 may perform a validation check to determine whether the quality of a set of FHR values that follow an accelerated FHR value in an ECG dataset 108a-n meets a quality threshold. For example, the fetal movement detection module 112 may evaluate the quality of a set of FHR values that follow in time in the ECG dataset 108a-n an accelerated FHR value marked as a possible accelerated FHR, and if the quality of the FHR values is below a quality threshold (e.g., below an FHR probability), then the fetal movement detection module 112 ends analysis for the possible accelerated FHR. As an illustration, the fetal movement detection module 112 may analyze five (5), ten (10), or fifteen (15) seconds worth of continuous FHR values that follow in time an accelerated FHR in an ECG dataset 108a-n, and if the quality of each FHR value is below the quality threshold, analysis of the possible accelerated FHR is ended. In the case that at least one of the FHR values is meets the quality threshold, then the fetal movement detection module 112 may flag the position of the possible accelerated FHR value as an identified accelerated FHR and output an indication that a fetal movement has been detected.

In yet another example, the fetal movement detection module 112 may perform a validation check to determine whether a time between an accelerated FHR value and a decelerated FHR value is a time that corresponds to a fetal movement. For example, after identifying a possible accelerated FHR value in an ECG dataset 108a-n as described above, the fetal movement detection module 112 may determine whether a duration of time between the position of the possible accelerated FHR value and the decelerated FHR value in the ECG dataset 108a-n is within a time bound (e.g., greater than 10 seconds and less than 5 minutes) that has been defined as a fetal movement. If the duration of time between the position of the possible accelerated FHR value and the decelerated FHR value is within the time bound, the fetal movement detection module 112 may flag the position of the possible accelerated FHR value as an identified accelerated FHR and output an indication that a fetal movement has been detected.

In some examples, the fetal movement detection module 112 may perform a combination of the validation checks described above. For example, after identifying a possible accelerated FHR value in an ECG dataset 108a-n, the fetal movement detection module 112 may (i) determine whether a quality of the accelerated FHR value and the FHR value meet a predetermined quality threshold, and (ii) determine whether the accelerated FHR value is followed in time by a subsequent accelerated FHR value, indicating that the accelerated FHR value is not an anomaly, and (iii) determine whether a time between an accelerated FHR value and a decelerated FHR value is a time that corresponds to a fetal movement. In the case that the validation checks are successful, fetal movement detection module 112 may output an indication that a fetal movement has been detected.

An ECG dataset 108a-n can contain multiple instances of FHR accelerations (e.g., increase or rise in FHR) and corresponding FHR decelerations (e.g., decrease or fall in FHR) that correlate to fetal movements. For example, the ECG dataset 108a-n is for a segment of time (e.g., 5, 8, 10 minutes) during which fetus movement can be detected. As such, after identifying a first instance of an FHR acceleration and corresponding FHR deceleration in an ECG dataset 108a-n that indicates a fetal movement, the fetal movement detection module 112 may continue analysis of FHR values in the ECG dataset 108a-n to determine whether a second instance of an FHR acceleration and corresponding FHR deceleration can be identified in the ECG dataset 108a-n. In some cases, analysis of an ECG dataset 108a-n may indicate that no fetal movement has been detected during the segment of time for which the ECG dataset 108a-n was collected.

In cases where the fetal movement detection module 112 identifies an accelerated FHR value in a first ECG dataset 108a, but is unable to identify a decelerated FHR value that corresponds to the accelerated FHR value in the first ECG dataset 108a, the fetal movement detection module 112 may set a continue analysis flag that allows the fetal movement detection module 112 to analyze a second ECG dataset 108b (i.e., the subsequent ECG dataset 108b in the memory buffer 106) to determine whether the second ECG dataset 108b contains a decelerated FHR value that corresponds to the accelerated FHR value in a first ECG dataset 108a. For example, when the fetal movement detection module 112 starts analysis of the second ECG dataset 108b, the fetal movement detection module 112 may determine whether the continue analysis flag is set and analyze the FHR values in the second ECG dataset 108b for a decelerated FHR value that corresponds to the accelerated FHR value identified in the first ECG dataset 108a. Accordingly, the fetal movement detection module 112 can identify an accelerated FHR that spans two or more ECG datasets 108a-n. In the event that an accelerated FHR is identified in the first ECG dataset 108a and a corresponding decelerated FHR is identified in the second ECG dataset 108b, the fetal movement detection module 112 may perform one or more of the validation checks described earlier (e.g., determine that the FHR values in the first and second ECG datasets 108a-b meet a quality threshold).

The various processes and/or other functionality of the processing system 100 may be executed on one or more processors that are in communication with one or more memory modules. The processing system 100 may include one or more computing devices. In some examples, the processing system 100 can include a data store (not shown) used to store ECG data and/or fetal heart rate probabilities output by the FHR extraction model 110. The term “data store” may refer to any device or combination of devices capable of storing, accessing, organizing and/or retrieving data. The storage system components of the data store may include storage systems such as volatile or non-volatile RAM, hard-drive type media, and a cloud storage network. The data store may be representative of a plurality of data stores as can be appreciated.

In some examples, the processing system 100 may include a network for transmitting data between servers, clients, and devices. The network may include any useful computing network, including an intranet, the Internet, a local area network, a wide area network, a wireless data network, or any other such network or combination thereof. Components utilized for such a system may depend at least in part upon the type of network and/or environment selected. Communication over the network may be enabled by wired or wireless connections and combinations thereof.

FIG. 1 illustrates that certain processing modules may be discussed in connection with this technology and these processing modules may be implemented as computing services. In one example configuration, a module may be considered a service with one or more processes executing on a server or other computer hardware. Such services may be centrally hosted functionality or a service application that may receive requests and provide output to other services or consumer devices. For example, modules providing services may be considered on-demand computing that are hosted in a server, virtualized service environment, grid or cluster computing system. An API may be provided for each module to enable a second module to send requests to and receive output from the first module. Such APIs may also allow third parties to interface with the module and make requests and receive output from the modules. While FIG. 1 illustrates an example of a system that may implement the techniques above, many other similar or different environments are possible. The example environments discussed and illustrated above are merely representative and not limiting.

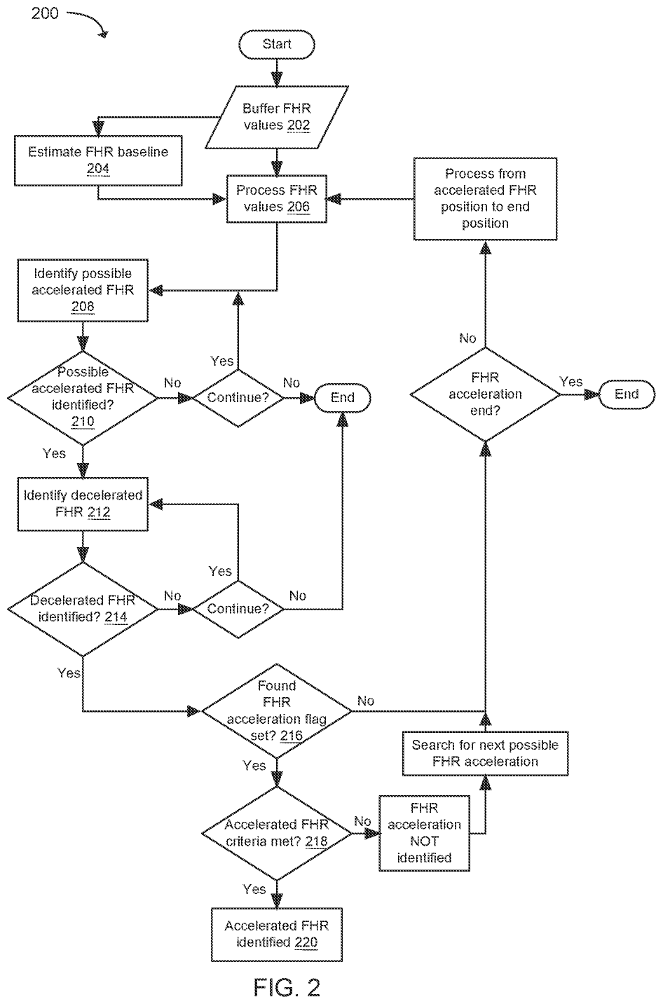

FIG. 2 is a flow diagram that illustrates an example method 200 for detecting fetal movement via analysis of a fetal heart rate to identify a fetal heart rate acceleration and corresponding deceleration that indicates a fetal movement. The method 200, as in block 202, can include buffering FHR values obtained from an ECG monitor in computer memory. The FHR values can be buffered in ECG datasets that correlate to a segment of time during which a fetal heart rate is monitored using the ECG monitor. In one example, a quality of an FHR value can be determined, and an FHR quality indicator or value can be stored with the FHR value in an ECG dataset. As a non-limiting example, a segment or block of time in which FHR values are collected can include five (5) minutes, eight (8) minutes, ten (10) minutes, or another defined block of time.

The FHR values included in an ECG dataset can be used to estimate an FHR baseline, as in block 204. In one example, the FHR values in the ECG dataset can be evaluated to determine whether a sufficient portion of the FHR values meet a quality threshold that allows the FHR baseline to be calculated. The quality threshold may be based on a probability (e.g., greater than zero) that a heart rate value in the ECG dataset is a fetal heart rate value. If the quality of the FHR values meets the quality threshold, the FHR baseline can be calculated. In the case that the quality of the FHR values does not meet the quality threshold, the ECG dataset may be discarded. In some examples, the FHR baseline can include a baseline offset.

As in block 206, the method 200 processes the FHR values in the ECG dataset to identify an accelerated FHR value. At the start of processing, an acceleration tracking flag used to indicate that a possible accelerated FHR has been identified may be initialized to false, and processing of the FHR values may start at a first position in the ECG dataset and continue to a last position in the ECG dataset.

As in block 208, identification of a possible accelerated FHR may include identifying an FHR value in the ECG dataset that exceeds the FHR baseline and is followed in time (relative to recording ECG data in the ECG dataset) by a corresponding decelerated FHR value that is less than or equal to the FHR baseline. In one example, additional conditions can be used to identify the possible accelerated FHR. These conditions can include (i) determining that an FHR value that immediately precedes the accelerated FHR value in the ECG dataset is less than or equal to the FHR baseline, and (ii) evaluating the quality of the accelerated FHR value and the FHR value that immediately precedes the accelerated FHR value in the ECG dataset to ensure that the quality meets a predetermined quality threshold. In the case that the conditions are met, the acceleration tracking flag can be set to true and an FHR quality counter can be initialized. Also, an acceleration found flag can be initialized to false to indicate that an accelerated FHR corresponding to a fetal movement has not yet been found.

As in block 210, in the case that a possible accelerated FHR is identified, as indicated by the acceleration tracking flag being set to true, then the method 200 in block 212 continues processing of the FHR values in the ECG dataset to identify a decelerated FHR that corresponds to the possible accelerated FHR. As each FHR value is processed, the quality of the FHR can be evaluated and if the quality of an FHR value meets the quality threshold, the FHR quality counter can be incremented.

Identification of a decelerated FHR may include identifying an FHR value in the ECG dataset that is less than or equal to the FHR baseline and that the quality of the FHR value meets a quality threshold. An additional condition can include determining that the quality of a set of FHR values that are greater than the FHR baseline located between the accelerated FHR value and the decelerated FHR value in the ECG dataset 108a-n meets a quality threshold. In the case that the conditions above are met, the acceleration found flag can be set to true.

As in block 214, in the case that a decelerated FHR is identified, then the method 200 in block 216 determines whether the acceleration found flag is set to true and, as in block 218, whether additional accelerated FHR criteria is met. The accelerated FHR criteria can include a requirement that (i) the quality of FHR values located between the possible accelerated FHR and the decelerated FHR meet the quality threshold, which can be determined by referencing the FHR quality counter, and that (ii) a time between the possible accelerated FHR and the decelerated FHR is within a time bound defined as a fetal movement (e.g., greater than 10 seconds and less than 5 minutes). In the case that the conditions above are met, then as in block 220, an accelerated FHR that corresponds to a fetal movement has been identified. As will be appreciated, the steps described above are one example of a method for detecting fetal movement based on identifying a fetal heart rate acceleration and corresponding deceleration that indicates a fetal movement. Many other similar or different steps are possible. The example steps discussed above are merely representative and not limiting.

FIG. 3 is a block diagram illustrating an example artificial neural network model 302 configured to extract a fetal heart rate from an ECG signal. In particular, the artificial neural network model 302 is one example of a machine learning algorithm that can be used by the FHR extraction module 110 (shown in FIG. 1) to analyze an ECG signal and predict an FHR value.

Previous methods for computing a fetal heart rate from abdominal electrodes identify R-peaks of a maternal ECG in an ECG signal and then create a template of the maternal ECG on each channel. The template of the maternal ECG is subtracted out of the ECG signal leaving a residual ECG signal containing the fetal ECG. Following the maternal ECG cancellation, some methods have used a source separation algorithm to attempt to group the fetal ECG signal into fewer channels. Following source separation, a peak detection method has been used to identity fetal R-peaks on each channel. After fetal R-peaks are identified, a channel selection algorithm has been used to choose the best peaks from each channel to produce the final R-peak locations. These R-peak locations have been used to compute the fetal heart rate. With the present technology described herein, there is no need to build a maternal template to cancel out a maternal ECG signal. There is also no need for source separation algorithms or a need to individually process channels.

The network architecture of the neural network model described herein provides improvements in the accuracy of FHR predictions obtained from an ECG signal over previous methods for computing a fetal heart rate from an ECG signal. In particular, the accuracy of FHR predictions output by the neural network model is improved by placing an FFT layer after a first series of convolutional layers and providing the output of the FFT layer to a dense layer of the neural network model. Placement of the FFT layer in this way improves the accuracy of FHR predictions by using the FFT layer to identify fundamental and harmonic frequencies of a fetal ECG signal, thereby reducing a number of parameters that are provided to the dense layer of the neural network model.

The artificial neural network model 302 (also referred to as the neural network model 302), in one example, can be an end-to-end neural network having an architecture that includes a series of convolution layers 306 followed by a fast Fourier transform (FFT) layer 308 and a dense decoding layer 310. As described in more detail below, ECG data 304 can be provided as input to the neural network model 302 and the architecture of the neural network model 302 can be configured to analyze the ECG data 304 to identify a fetal ECG signal and process the fetal ECG signal to generate an FHR probability 312.

As illustrated, the architecture of the neural network model 302 includes a series of convolution layers 306. The series of convolution layers 306 can include any number of convolution layers. In a specific example of the architecture of the neural network model 302, the series of convolutional layers 306 can include three convolutional layers. The convolution layers 306 can be configured to separate a maternal ECG signal from a fetal ECG signal. An ECG signal obtained from the maternal abdomen of a pregnant subject includes ECG information for both the subject and the subject's fetus. As an illustration, electrodes can be attached to the maternal abdomen of a pregnant subject and the electrical signals of the subject's and the fetus' hearts can be recorded. Consequently, the ECG signal obtained from the maternal abdomen of the pregnant subject includes artifacts of a maternal ECG signal. The convolution layers 306 of the neural network model 302 can be configured to remove the maternal ECG signal artifacts (as well as other artifacts) from the ECG signal to better isolate a fetal ECG signal.

In some examples, the series of convolutional layers 306 may be a first convolutional layer that proceeds the FFT layer 308, and the architecture of the neural network model 302 can include a second series of convolutional layers (not shown) located between the FFT layer 308 and the dense decoding layer 310. The second series of convolutional layers is similar to the first but acts on the frequency transformed signal allowing the signal to be cleaned in the frequency domain potentially improving signal quality. For example, the second series of convolutional layers can be used to identify and remove artifacts from a Fourier transform output by the FFT layer 308, which may further improve a fetal ECG signal.

The architecture of the neural network model 302 shown in FIG. 3 places the FFT layer 308 between the convolution layer 306 and the dense decoding layer 310. The FFT layer 308 can be configured to apply a fast Fourier transform technique to a fetal ECG signal output by the series of convolution layers 306 to convert the fetal ECG signal to a representation of a fundamental frequency and harmonic frequencies. Applying a fast Fourier transform technique to a fetal ECG signal allows resulting fetal ECG frequencies to be quantized into values that can be classified into heart rate values.

Placing the FFT layer 308 after the series of convolution layers 306 and before the dense decoding layer 310 improves performance of predicting fetal heart rates using the neural network model 302. In particular, applying a fast Fourier transform technique to a fetal ECG signal output by the series of convolution layers 306 reduces a number of parameters that are provided to the dense decoding layer 310 of the neural network model 302. By reducing the number of parameters provided as input to the dense decoding layer 310, an amount of data processed by the dense decoding layer 310 is decreased, which results in a shorter amount of time to generate FHR probabilities 312.

Also, placing the FFT layer 308 after the series of convolution layers 306 and before the dense decoding layer 310 improves accuracy of FHR predictions output by the neural network model 302. More specifically, applying a fast Fourier transform technique to fetal ECG signals output by the series of convolution layers 306 allows fetal ECG frequencies to be quantized, thereby restricting a number of possible fetal ECG frequency values that can be classified as heart rate values. This allows for the use of classification, as opposed to using regression, which can produce bias in the ECG data. As an example, using a means squared error technique as a loss function tends to pull values toward the mean, which creates bias in the ECG data. Using a fast Fourier transform technique reduces the chance of bias in the ECG data. For example, a fast Fourier transform technique allows fetal ECG frequencies output by the FFT layer 308 to be classified as a probability distribution of fetal heart rates, and allows for a maximum likelihood estimation to be applied to the probability distribution of fetal heart rates to determine an FHR probability 312.

The dense decoding layer 310 included in the neural network model 302 architecture can be configured to decode ECG frequency representations output by the FFT layer 308 into FHR predictions. In one example, the dense decoding layer 310 decodes the ECG frequency representations into fetal heart rate information (e.g., Beats Per Minute (BPM)) used to generate an FHR prediction. As an example, the dense decoding layer 310 selects an ECG frequency representation (e.g., a harmonic frequency) output by the FFT layer 308 and applies a mask to the ECG frequency representation which is used to visualize the ECG frequency representation as a fetal heart rate value (e.g., 131, 132, or 133 BPM). Thereafter, the FHR values can be scored to create a probability distribution that indicates a maximum likelihood of a fetal heart rate, which can be output as an FHR probability 312. In one example, after scoring the FHR values, the FHR values can be input to a softmax layer (not shown) that has one neuron node for each FHR value. The softmax layer can normalize the FHR values to sum to a value of 1, creating a probability distribution of FHR values that indicates a maximum likelihood of an FHR value.

The following example is an illustration of an end-to-end artificial neural network architecture configured to generate FHR probabilities based on ECG data input. As will be appreciated, the example artificial neural network architecture shown in Example 1 is merely representative of a neural network architecture and is not meant to be limiting.

The neural network model 302 can be trained to generate FHR probabilities using a training dataset of ECG data. The training data set can comprise ECG data collected from a number of pregnant subjects using an ECG monitor. The ECG data can be obtained using ECG electrodes collected, for example, at 1 kHz. In one example, an ultrasound monitor can be used to acquire a fetal heart rate reference from one or more pregnant subjects. The ECG data can be low pass filtered and down sampled (e.g., to 200 Hz). The ECG data can then be split into a training dataset and a test dataset. In one example, the neural network model 302 can be trained using categorical cross entropy to label ECG data in the training dataset to a heart rate category and an Adam optimizer to update weights assigned to the ECG data. As will be appreciated, techniques other than those described above can be used to train the neural network model 302.

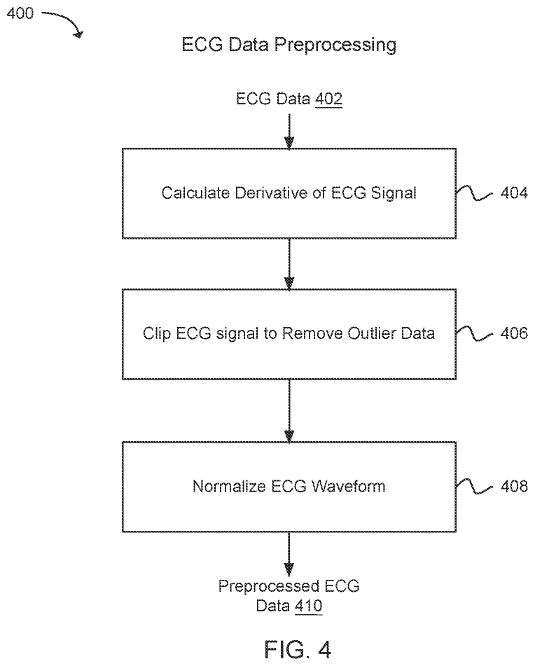

FIG. 4 is a flow diagram that illustrates an example method 400 for preprocessing ECG data. Prior to inputting ECG data 402 to the neural network model described above, the ECG data 402 can be preprocessed to format the ECG data 402 for input to the neural network model. Preprocessing can be performed prior to training the neural network model, and prior to inference time in which a predicted FHR value is generated using the trained neural network model.

Preprocessing of ECG data 402 can include one or more preprocessing steps. In one example, the preprocessing steps can include: (i) calculating a derivative of an ECG signal to accentuate high frequency components in the ECG signal, (ii) clipping the ECG signal to remove outlier data included in the ECG data, and (iii) normalizing an ECG waveform of the ECG signal to a standard deviation.

As in block 404, the preprocessing step of taking a derivative of an ECG signal included in ECG data 402 can be performed to accentuate high frequency components of a fetal QRS complex (combination of three of the graphical deflections seen on a typical electrocardiogram) in the ECG signal. This step can be performed to emphasize the QRS complex in the ECG data so that the information is more apparent when processed by the neural network model.

As in block 406, the preprocessing step of clipping an ECG signal included in ECG data 402 can be performed to remove outlier data included in the ECG data 402. Outliers can be caused by either the maternal QRS complex or maternal movement. In one example, clipping an ECG signal can include computing amplitude percentiles of the ECG signal and clipping values that are greater than a difference between amplitude percentiles. As an illustration, the 25th, 50th, and 75th amplitude percentiles of an ECG signal can be computed, and values can be clipped that are greater than six (6) times the difference between the 50th and 75th percentile of less than six (6) times the difference between the 50th percentile and the 25th percentile.

As in block 408, the preprocessing step of normalizing an ECG waveform of an ECG signal to a standard deviation can be performed to ensure that ECG signals included in ECG data 402 are approximately the same scale. After preprocessing ECG data, the preprocessed ECG data 410 can be provided as input to the neural network model to generate FHR probabilities as described earlier.

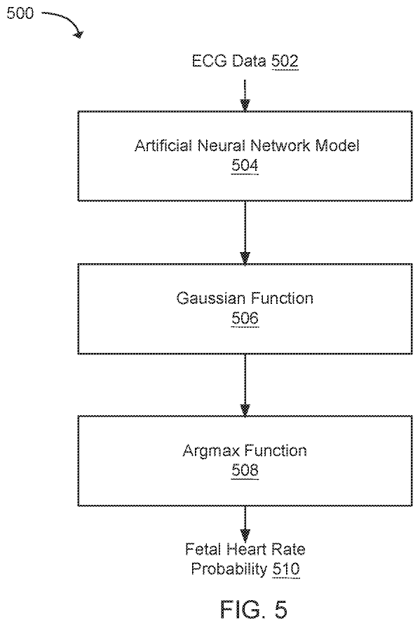

FIG. 5 is a block diagram illustrating an example of a processing system 500 that incorporates prior FHR predictions to determine an FHR probability 510. The processing system 500 can be used by the FHR extraction module 110 shown in FIG. 1. The processing system 500 can incorporate prior FHR predictions into current predictions of fetal heart rates at inference time. The prior FHR predictions can be used to gauge whether a current prediction is reasonable. For example, a sudden change in a fetal heart rate is rare. Therefore, a prior FHR prediction can be compared to a current heart rate prediction to determine whether a change in heart rate from the prior FHR prediction to the current prediction is a data anomaly or is reasonable. Accordingly, a current heart rate prediction that is close to a previous heart rate prediction can be treated as being more likely accurate than current heart rate predictions that are far away from previous heart rate predictions.

In the example illustrated in FIG. 5, the processing system 500 can include an artificial neural network model 504. The artificial neural network model 504 can have the architecture described earlier in association with FIG. 1. In addition to the artificial neural network model 504, the processing system 500 can include a Gaussian function 506 and an argmax function 508. One or more prior FHR predictions can be incorporated into a current FHR prediction at inference time (i.e., at the time the trained neural network model 504 is used to generate an FHR probability) by multiplying a probability distribution of fetal heart rates output by the artificial neural network model 504 by the Gaussian function 506. In one example, the Gaussian function 506 can have a mean value that is equal to the previous heart rate prediction. As an illustration, a fetal heart rate distribution can be multiplied by the Gaussian function 506 with a standard deviation of 10 BPM and a mean value equal the most recent heart rate prediction. In one example, if no recent heart rate prediction is available, the Gaussian function 506 can be replaced with an identity vector. The identity vector can be a vector of one (1) values of the same length as the Gaussian vector, resulting in not modifying the input to the argmax function 508 described below. In another example where no recent heart rate prediction is available, the Gaussian function 506 step described above may not be performed, and the argmax function 508 described below can be applied to the probability distribution.

After multiplying the probability distribution by the Gaussian function 506, an argmax function 508 (arguments of the maxima) can be used to produce the FHR probability 510. For example, an argmax of the heart rate distribution can be calculated to produce the FHR probability 510. The probability of the heart rate value can be used as a quality indicator for FHR values. Fetal heart rates with low probability may be rejected to avoid false heart rate readings.

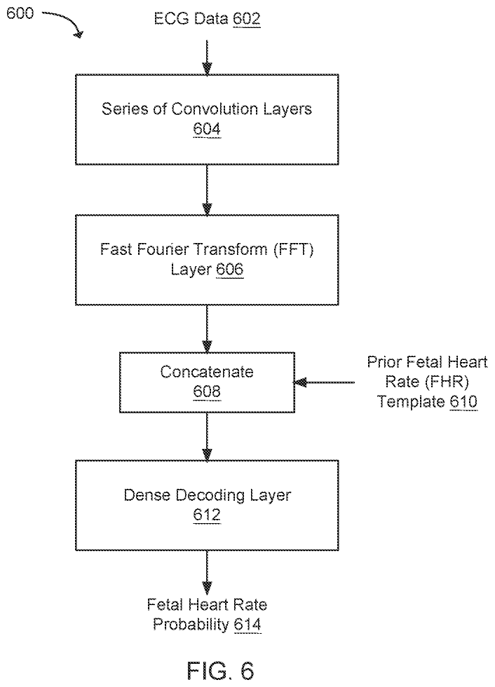

FIG. 6 is a block diagram that illustrates an example network architecture for an artificial neural network model 600, which can be used by the FHR extraction module 110 shown in FIG. 1, to incorporate prior FHR information to generate an FHR probability 614. In the discussion above of the processing system 500 shown in FIG. 5, prior FHR information is incorporated after generating a probability distribution of fetal heart rates. The network architecture of the artificial neural network model 600 shown in FIG. 6 incorporates prior FHR information into the neural network model 600 to allow training of the neural network model 600 to include the prior FHR information.

A prior FHR prediction can be used as part of generating a current FHR probability in a number of ways. In one example, a series of sine waves corresponding to a fundamental frequency and harmonic frequencies of a prior FHR prediction can be summed. The resulting sum provides a prior FHR template 610 that can be passed to the neural network model 600. One method that can be used to pass a prior FHR template 610 to the neural network model 600 includes applying a Fourier transform (e.g., Fast Fourier Transform (FFT)) to the prior FHR template 610, forming a prior FHR transform, and concatenating the prior FHR transform to the Fourier transform output by the FFT layer 606 of the neural network model 600.

As an illustration, ECG data 602 included in a training dataset can be input to a series of convolution layers 604 to separate a fetal ECG signal from a maternal ECG signal. The FFT layer 606 can be applied to the fetal ECG signal to produce a Fourier transform of the fetal ECG signal. A Fourier transform of a prior FHR template 610 can be produced, and the Fourier transform of the prior FHR template 610 can be concatenated 608 to the Fourier transform of the fetal ECG signal. The resulting concatenated Fourier transform comprising ECG frequency representations of the ECG data 602 and the prior FHR template 610 can be input to a dense decoding layer 612 of the neural network model 600. The dense decoding layer 612 decodes the ECG frequency representations, as described earlier, and outputs an FHR probability 614.

The following example illustrates an end-to-end artificial neural network architecture configured to incorporate prior FHR information into an artificial neural network model to generate an FHR probability. As will be appreciated, the example artificial neural network architecture shown in Example 2 is merely representative of a neural network architecture and is not limiting.

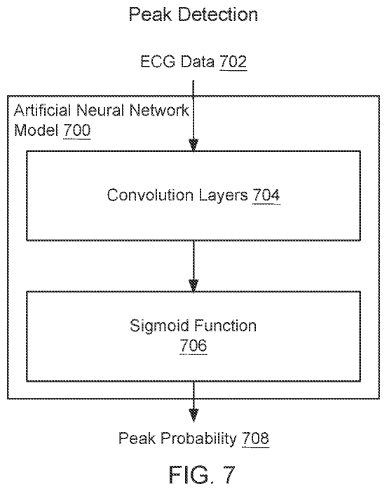

FIG. 7 is a block diagram that illustrates an example artificial neural network model 700 configured to generate peak probabilities 708 that correspond to individual heart rate peaks which can be decoded to produce a final FHR prediction. For example, peak probabilities 708 can be decoded using peak detection to produce a final heart rate probability. In one example, a peak detection method can use the preprocessing technique described above in association with FIG. 2 to preprocess ECG data 702. Following preprocessing, a waveform can be passed to the neural network model 700 in short windows (e.g., 100, 125, or 150 samples). The neural network model 700 can include of a series of convolutional layers 704 with a sigmoid function 706 output corresponding to a peak probability 708. Example 3 below illustrates an artificial neural network architecture configured to generate a peak probability. As will be appreciated, the example artificial neural network architecture shown in Example 3 is merely representative and is not meant to be limiting.

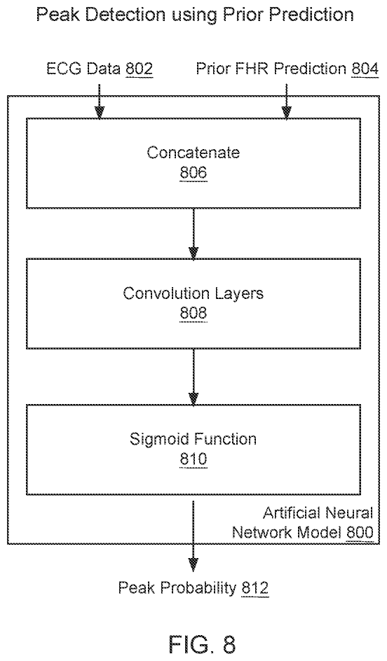

FIG. 8 is a block diagram illustrating an example artificial neural network model 800 trained using prior FHR predictions. The neural network model 800 can be trained using binary cross entropy, mean squared error, least absolute deviation, or another appropriate loss function. The neural network model 800 can include a concatenate layer 806, a series of convolution layers 808, and a sigmoid function 840 layer. After peak probabilities have been generated, the peak probabilities can be decoded into peak locations and fetal heart rate using a variety of known peak detection methods. The neural network model 800 can be provided with information from prior FHR predictions (prior prediction 804). For example, the expected location of the next peak can be passed to the neural network model 800 in addition to ECG data 802. One example of passing prior FHR predictions 804 to the neural network model 800 is to add an additional input channel of the same length as the ECG signal with a one (1) where the next fetal heart rate is expected to be and zero (0) otherwise. The neural network model 800 can learn to incorporate the prior FHR prediction 804 to improve prediction accuracy. Example 4 below illustrates passing prior FHR prediction information to neural network model. As described earlier, the peak probabilities can be decoded into peak locations and a fetal heart rate using a variety of known peak detection methods.

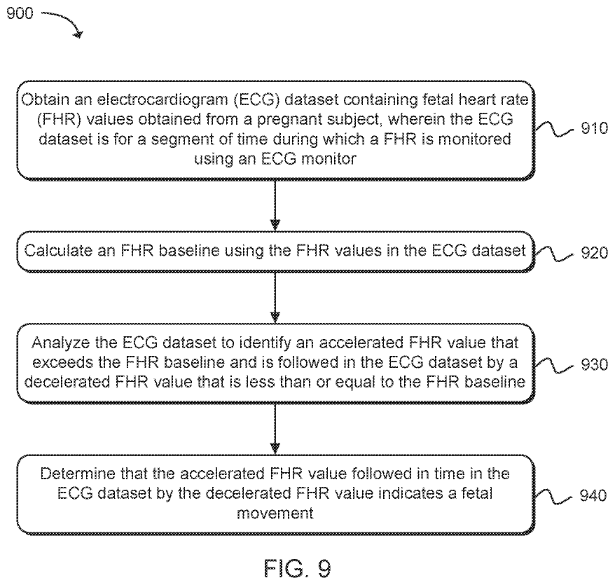

FIG. 9 is a flow diagram illustrating an example method 900 for determining fetal movements by analyzing a fetal heart rate to identify a FHR acceleration and corresponding deceleration indicating fetal movement.

As in block 910, an electrocardiogram (ECG) dataset containing FHR values may be obtained. The ECG dataset can be acquired from a pregnant subject wearing an ECG monitor. The ECG dataset may be for a segment of time during which a FHR is monitored using the ECG monitor. In one example, an artificial neural network model can be trained to predict the FHR values using a training ECG dataset, wherein the artificial neural network model can include a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to fetal heart rate predictions.

As in block 920, an FHR baseline can be calculated using the FHR values in the ECG dataset. In one example, a quality of each FHR value in the ECG dataset can be determined, and the quality of the FHR values contained in the ECG dataset can be evaluated to determine whether a sufficient portion of the FHR values meets a quality threshold that allows an FHR baseline to be calculated. The quality of an FHR value may be based on a probability (e.g., greater than zero) that the heart rate value is a fetal heart rate value. In the case that a sufficient portion of the FHR values meets the quality threshold, the FHR baseline can be calculated. In one example, a baseline offset can be added to the FHR baseline.

As in block 930, the ECG dataset can be analyzed to identify an accelerated FHR value that exceeds the FHR baseline and is followed by a decelerated FHR value that is less than or equal to the FHR baseline. In one example, identifying an accelerated FHR can include determining that an accelerated FHR value is greater than the FHR baseline plus the FHR baseline offset.

In one example, identifying the accelerated FHR can also include determining that a FHR value that immediately precedes the accelerated FHR value in the ECG dataset is less than or equal to the FHR baseline plus the FHR baseline offset.

In one example, identifying the accelerated FHR can also include determining that a quality of the accelerated FHR value and the FHR value that immediately precedes the accelerated FHR value in the ECG dataset meet a predetermined quality threshold.

In one example, identifying the accelerated FHR can also include marking a position of the accelerated FHR value in the ECG dataset as a possible accelerated FHR, evaluating a quality of FHR values located between the accelerated FHR value and the decelerated FHR value, determining that a sufficient portion of the FHR values between the accelerated FHR value and the decelerated FHR value meet a predetermined quality threshold, and flagging the position of the accelerated FHR value in the ECG dataset as an identified accelerated FHR.

In one example, identifying the accelerated FHR can also include identifying a subsequent accelerated FHR value in the ECG dataset that is within a time threshold of the position of the accelerated FHR value in the ECG dataset.

In one example, identifying the accelerated FHR can also include determining that a duration of time between the accelerated FHR value and the decelerated FHR value in the ECG dataset is within a time bound defined as a fetal movement.

In one example, identifying the accelerated FHR can also include setting a continue analysis flag when a decelerated FHR value that corresponds to the accelerated FHR value cannot be identified in the ECG dataset, and analyzing a subsequent ECG dataset to identify the decelerated FHR value in the subsequent ECG dataset.

As in block 940, in the case that the conditions described above are met, a determination can be made that the accelerated FHR value followed in time by the decelerated FHR value in the ECG dataset indicates a fetal movement.

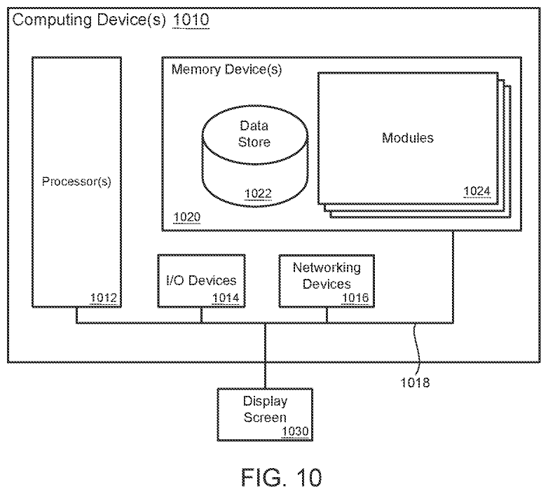

FIG. 10 illustrates a computing device 1010 on which modules of this technology may execute. A computing device 1010 is illustrated on which a high-level example of the technology may be executed. The computing device 1010 may include one or more processors 1012 that are in communication with memory devices 1020. The computing device 1010 may include a local communication interface 1018 for the components in the computing device. For example, the local communication interface 1018 may be a local data bus and/or any related address or control busses as may be desired.

The memory device 1020 may contain modules 1024 that are executable by the processor(s) 1012 and data for the modules 1024. The modules 1024 can include an FHR extraction module, convolution modules, fast Fourier transform modules, dense decoding modules, a fetal movement detection module, and other modules. The modules 1024 may execute the functions described earlier. A data store 1022 may also be located in the memory device 1020 for storing data related to the modules 1024 and other applications along with an operating system that is executable by the processor(s) 1012.

Other applications may also be stored in the memory device 1020 and may be executable by the processor(s) 1012. Components or modules discussed in this description that may be implemented in the form of software using high-level programming languages that are compiled, interpreted or executed using a hybrid of the methods.

The computing device 1010 may also have access to I/O (input/output) devices 1014 that are usable by the computing device 1010. One example of an I/O device is a display screen 1030 that is accessible to the computing device 1010. Networking devices 1016 and similar communication devices may be included in the computing device. The networking devices 1016 may be wired or wireless networking devices that connect to the internet, a LAN, WAN, or other computing network.

The components or modules that are shown as being stored in the memory device 1020 may be executed by the processor(s) 1012. The term “executable” may mean a program file that is in a form that may be executed by a processor 1012. For example, a program in a higher level language may be compiled into machine code in a format that may be loaded into a random access portion of the memory device 1020 and executed by the processor 1012, or source code may be loaded by another executable program and interpreted to generate instructions in a random access portion of the memory to be executed by a processor. The executable program may be stored in any portion or component of the memory device 1020. For example, the memory device 1020 may be random access memory (RAM), read only memory (ROM), flash memory, a solid-state drive, memory card, a hard drive, optical disk, floppy disk, magnetic tape, or any other memory components.

The processor 1012 may represent multiple processors and the memory device 1020 may represent multiple memory units that operate in parallel to the processing circuits. This may provide parallel processing channels for the processes and data in the system. The local communication interface 1018 may be used as a network to facilitate communication between any of the multiple processors and multiple memories. The local communication interface 1018 may use additional systems designed for coordinating communication such as load balancing, bulk data transfer and similar systems.

While the flowcharts presented for this technology may imply a specific order of execution, the order of execution may differ from what is illustrated. For example, the order of two more blocks may be rearranged relative to the order shown. Further, two or more blocks shown in succession may be executed in parallel or with partial parallelization. In some configurations, one or more blocks shown in the flow chart may be omitted or skipped. Any number of counters, state variables, warning semaphores, or messages might be added to the logical flow for purposes of enhanced utility, accounting, performance, measurement, troubleshooting or for similar reasons.

Some of the functional units described in this specification have been labeled as modules, in order to more particularly emphasize their implementation independence. For example, a module may be implemented as a hardware circuit comprising custom VLSI circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in software for execution by various types of processors. An identified module of executable code may, for instance, comprise one or more blocks of computer instructions, which may be organized as an object, procedure, or function. Nevertheless, the executables of an identified module need not be physically located together, but may comprise disparate instructions stored in different locations which comprise the module and achieve the stated purpose for the module when joined logically together.

Indeed, a module of executable code may be a single instruction, or many instructions and may even be distributed over several different code segments, among different programs and across several memory devices. Similarly, operational data may be identified and illustrated herein within modules and may be embodied in any suitable form and organized within any suitable type of data structure. The operational data may be collected as a single data set, or may be distributed over different locations including over different storage devices. The modules may be passive or active, including agents operable to perform desired functions.

The technology described here may also be stored on a computer readable storage medium that includes volatile and non-volatile, removable and non-removable media implemented with any technology for the storage of information such as computer readable instructions, data structures, program modules, or other data. Computer readable storage media include, but is not limited to, a non-transitory machine-readable storage medium, such as RAM, ROM, EEPROM, flash memory or other memory technology, CD-ROM, digital versatile disks (DVD) or other optical storage, magnetic cassettes, magnetic tapes, magnetic disk storage or other magnetic storage devices, or any other computer storage medium which may be used to store the desired information and described technology.

The devices described herein may also contain communication connections or networking apparatus and networking connections that allow the devices to communicate with other devices. Communication connections are an example of communication media. Communication media typically embodies computer readable instructions, data structures, program modules and other data in a modulated data signal such as a carrier wave or other transport mechanism and includes any information delivery media. A “modulated data signal” means a signal that has one or more of its characteristics set or changed in such a manner as to encode information in the signal. By way of example and not limitation, communication media includes wired media such as a wired network or direct-wired connection and wireless media such as acoustic, radio frequency, infrared and other wireless media. The term computer readable media as used herein includes communication media.

Reference was made to the examples illustrated in the drawings and specific language was used herein to describe the same. It will nevertheless be understood that no limitation of the scope of the technology is thereby intended. Alterations and further modifications of the features illustrated herein and additional applications of the examples as illustrated herein are to be considered within the scope of the description.

Furthermore, the described features, structures, or characteristics may be combined in any suitable manner in one or more examples. In the preceding description, numerous specific details were provided, such as examples of various configurations to provide a thorough understanding of examples of the described technology. It will be recognized, however, that the technology may be practiced without one or more of the specific details, or with other methods, components, devices, etc. In other instances, well-known structures or operations are not shown or described in detail to avoid obscuring aspects of the technology.

Although the subject matter has been described in language specific to structural features and/or operations, it is to be understood that the subject matter defined in the appended claims is not necessarily limited to the specific features and operations described above. Rather, the specific features and acts described above are disclosed as example forms of implementing the claims. Numerous modifications and alternative arrangements may be devised without departing from the spirit and scope of the described technology.

1. A system for determining fetal movement, comprising:
at least one processor;
a memory device including instructions that, when executed by the at least one processor, cause the system to:
obtain an electrocardiogram (ECG) dataset containing fetal heart rate (FHR) values acquired from a pregnant subject, wherein the ECG dataset is for a segment of time during which an FHR is monitored using an ECG monitor;
calculate an FHR baseline using the FHR values in the ECG dataset;
analyze the ECG dataset to identify an accelerated FHR value that exceeds the FHR baseline and a decelerated FHR value that is less than or equal to the FHR baseline, wherein the accelerated FHR value occurs in the ECG dataset before the decelerated FHR value; and
determine that the accelerated FHR value followed in time in the ECG dataset by the decelerated FHR value indicates a fetal movement.

2. The system in claim 1, wherein the memory device further includes instructions that, when executed by the at least one processor, cause the system to:
determine a quality of each FHR value in the ECG dataset to represent an FHR; and
evaluate the quality of the FHR values contained in the ECG dataset to determine that a sufficient portion of the FHR values meet a quality threshold that allows the FHR baseline to be calculated.

3. The system in claim 1, wherein the instructions that, when executed by the at least one processor, cause the system to analyze the ECG dataset to identify an accelerated FHR value further cause the system to:
determine that the accelerated FHR value is greater than the FHR baseline plus an FHR baseline offset; and
determine that an FHR value that immediately precedes the accelerated FHR value in the ECG dataset is less than or equal to the FHR baseline plus the FHR baseline offset.

4. The system in claim 3, wherein the memory device further includes instructions that, when executed by the at least one processor, cause the system to:
determine that a quality of the accelerated FHR value and the FHR value that immediately precedes the accelerated FHR value in the ECG dataset meet a predetermined quality threshold.

5. The system in claim 1, wherein the memory device further includes instructions that, when executed by the at least one processor, cause the system to:
mark a position of the accelerated FHR value in the ECG dataset as a possible accelerated FHR;
evaluate a quality of the FHR values located between the accelerated FHR value and the decelerated FHR value;
determine that a sufficient portion of the FHR values between the accelerated FHR value and the decelerated FHR value meet a predetermined quality threshold; and
flag the position of the accelerated FHR value in the ECG dataset as an identified accelerated FHR.

6. The system in claim 5, wherein the memory device further includes instructions that, when executed by the at least one processor, cause the system to:
continue processing of the ECG dataset to identify a subsequent accelerated FHR value in the ECG dataset that is within a time threshold of the position of the accelerated FHR value in the ECG dataset; and
flag the position of the accelerated FHR value in the ECG dataset as an identified accelerated FHR when the subsequent accelerated FHR value is identified.

7. The system in claim 5, wherein the memory device further includes instructions that, when executed by the at least one processor, cause the system to:
determine that a duration of time between the accelerated FHR value and the decelerated FHR value in the ECG dataset is within a time bound defined as a fetal movement.

8. The system in claim 1, wherein the memory device further includes instructions that, when executed by the at least one processor, cause the system to:
set a continue analysis flag when a potential accelerated FHR value is located in a first ECG dataset and a corresponding decelerated FHR value, which together with the potential accelerated FHR value indicate fetal movement, is not identified in the first ECG dataset;
determine that the continue analysis flag is set; and
analyze a second ECG dataset that follows the first ECG dataset in time to identify the corresponding decelerated FHR value in the second ECG dataset.

9. The system in claim 1, wherein the memory device further includes instructions that, when executed by the at least one processor, cause the system to:
train an artificial neural network model to predict the FHR values using a training ECG dataset, wherein the artificial neural network model includes a first series of convolutional layers to separate a fetal ECG signal from a maternal ECG signal, a fast Fourier transform (FFT) layer to convert the fetal ECG signal to an ECG frequency representation, and a dense layer to decode the ECG frequency representation to a FHR prediction.

10. The system in claim 9, wherein the memory device further includes instructions that, when executed by the at least one processor, cause the system to:
generate the ECG dataset using the artificial neural network model, wherein ECG data generated by the ECG monitor is input to the artificial neural network model, and the ECG dataset is generated from FHR predictions output by the artificial neural network model.

11. The system in claim 9, wherein the artificial neural network model is trained using categorical cross entropy to label the ECG data in the training dataset to a heart rate category and an Adam optimizer to update weights assigned to the ECG data.

12. The system in claim 9, wherein the memory device further includes instructions that, when executed by the at least one processor, cause the system to preprocess the ECG data, wherein preprocessing includes (i) calculating a derivative of the ECG signal to accentuate high frequency components of a fetal QRS complex in the ECG data, (ii) clipping the ECG signal to remove outlier data included in the ECG data, and (iii) normalizing an ECG waveform of the ECG signal to a standard deviation.

13. The system in claim 9, wherein the memory device further includes instructions that, when executed by the at least one processor, cause the system to:
generate a prior FHR template by summing a series of sine waves that correspond to a fundamental frequency of a prior FHR prediction and a harmonic of the prior FHR prediction; and
input the prior FHR template to the artificial neural network model during training of the artificial neural network model.

14. The system in claim 13, wherein inputting the prior FHR prediction to the artificial neural network model during training further comprises:
applying a Fourier transform to the prior FHR template to form a prior FHR transform;
obtaining an FHR transform output by the FFT layer of the artificial neural network model;
concatenating the prior FHR transform to the FHR transform output by the FFT layer to form a concatenated FHR transform; and
providing the concatenated FHR transform to the dense layer of the artificial neural network model.

15. The system in claim 9, wherein an output layer of the neural network model is a softmax layer that has a neuron node for each fetal heart rate value.

16. The system in claim 9, further comprising:
generating a heart rate distribution, wherein a current FHR prediction is multiplied by a Gaussian function that has a mean value that is equal to a prior FHR prediction; and
calculating an argmax of the heart rate distribution to produce the FHR prediction.

17. A computer implemented method for determining fetal movement, comprising:
obtaining electrocardiogram (ECG) data from a pregnant subject, wherein the ECG data contains maternal and fetal heart rate information;
inputting the ECG data to an artificial neural network model trained to predict which heart rate heart rate values in the ECG data are fetal heart rate (FHR) values, wherein the artificial neural network model includes a first series of convolutional layers to separate a fetal ECG signal from a maternal ECG signal, a fast Fourier transform (FFT) layer to convert the fetal ECG signal to ECG frequency representations, and a dense layer to decode the ECG frequency representations to FHR predictions;
generating an ECG dataset of FHR values from the FHR predictions output by the artificial neural network model;
calculating an FHR baseline using the FHR values in the ECG dataset; and
analyzing the ECG dataset to identify an indication of fetal movement defined by an accelerated FHR value which is followed in time by a decelerated FHR value in the ECG dataset, wherein the accelerated FHR value exceeds the FHR baseline, and the decelerated FHR value is less than or equal to the FHR baseline.

18. The computer implemented method in claim 17, further comprising training the artificial neural network model using a training ECG dataset of ECG data collected from a plurality of pregnant subjects using an ECG monitor, wherein the artificial neural network model is trained using categorical cross entropy to label the ECG data in the training dataset to a heart rate category and an Adam optimizer to update weights assigned to the ECG data.

19. The computer implemented method in claim 17, further comprising preprocessing the ECG data, wherein preprocessing includes (i) calculating a derivative of the ECG signal to accentuate high frequency components of a fetal QRS complex in the ECG data, (ii) clipping the ECG signal to remove outlier data included in the ECG data, and (iii) normalizing an ECG waveform of the ECG signal to a standard deviation.

20. The computer implemented method in claim 17, wherein analyzing the ECG dataset to identify an indication of fetal movement further comprises:
determining that the accelerated FHR value is greater than the FHR baseline plus an FHR baseline offset;
determining that an FHR value that immediately precedes the accelerated FHR value in the ECG dataset is less than or equal to the FHR baseline plus the FHR baseline offset and meet a predetermined quality threshold; and
determining that a sufficient portion of intervening FHR values located between the accelerated FHR value and the decelerated FHR value meet a predetermined quality threshold.

21.-23. (canceled)
